(19) United States
(12) Patent Application Publication
(10) Pub. No.: US 2026/0284081 A1
(43) Pub. Date: Sep. 24, 2026

(54) METHOD FOR TREATING OR PREVENTING AN AGING-RELATED CONDITION

(71) Applicants: National Yang Ming Chiao Tung University, Hsinchu City (TW); TAIPEI MEDICAL UNIVERSITY, Taipei City (TW)

(72) Inventors: Ting-Ting Chang, Taipei City (TW); Jaw-Wen Chen, Taipei City (TW)

(73) Assignees: National Yang Ming Chiao Tung University, Hsinchu City (TW); TAIPEI MEDICAL UNIVERSITY, Taipei City (TW)

(21) Appl. No.: 19/571,445

(22) Filed: Mar. 18, 2026

Related U.S. Application Data
(60) Provisional application No. 63/774,725, filed on Mar. 19, 2025.

(51) Int. Cl. A61K 31/58; A61P 9/00

(52) U.S. Cl. CPC A61K 31/58; A61P 9/00

(57) ABSTRACT

The present disclosure provides a method for treating or preventing an aging-related condition. The method for treating or preventing an aging-related condition includes: administering to a subject in need thereof a therapeutically effective amount of diosgenin. The aging-related condition is selected from the group consisting of vascular dysfunction, dermal dysfunction, and metabolic dysfunction.

### CROSS REFERENCE TO RELATED APPLICATIONS

This application claims the benefit of U.S. Provisional Application No. 63/774,725, filed on Mar. 19, 2025. The content of the application is incorporated herein by reference.

### BACKGROUND OF THE INVENTION

### 1. Field of the Invention

The present disclosure relates to chemokine inhibition and anti-inflammatory therapeutics, and particularly, to a small-molecule inhibitor of C-C motif chemokine ligand 4 (CCL4) for a therapeutic strategy for aging-related vascular, dermal, and metabolic dysfunction.

### 2. Description of the Prior Art

Aging is associated with progressive functional decline in multiple tissues and organ systems. Among the biological processes implicated in aging is chronic low-grade inflammation, which has been correlated with vascular endothelial dysfunction, impaired angiogenesis, delayed wound healing, and metabolic dysregulation.

Chemokines are signaling proteins involved in inflammatory regulation and immune cell recruitment. C-C motif chemokine ligand 4 (CCL4), also known as macrophage inflammatory protein-1β (MIP-1β), has been implicated in inflammatory signaling pathways, including activation of nuclear factor kappa-B (NF-κB) and induction of pro-inflammatory mediators such as interleukin-6 (IL-6) and tumor necrosis factor-α (TNF-α). Elevated expression of CCL4 has been observed under certain inflammatory conditions

Strategies aimed at modulating inflammatory pathways have been investigated in various contexts. However, approaches specifically directed to directly modulating CCL4 activity using small-molecule compounds remain limited. In particular, identification of small molecules capable of directly interacting with CCL4 to attenuate its activity has not been clearly established.

Accordingly, there is an unmet remain in the art to identify small-molecule compounds capable of directly interacting with CCL4 and modulating its activity, thereby providing potential therapeutic approaches for aging-related vascular, dermal, and metabolic dysfunction.

### SUMMARY OF THE INVENTION

To solve the aforementioned problems, the present disclosure provides a method for treating or preventing an aging-related condition, including: administering to a subject in need thereof a therapeutically effective amount of diosgenin. The aging-related condition may be selected from the group consisting of vascular dysfunction, dermal dysfunction, and metabolic dysfunction.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A illustrates emission spectra of CCL4 in the presence of diosgenin according to an embodiment of the present disclosure. The interaction results reveal that diosgenin may quench the intrinsic fluorescence of CCL4 in a dose dependent manner

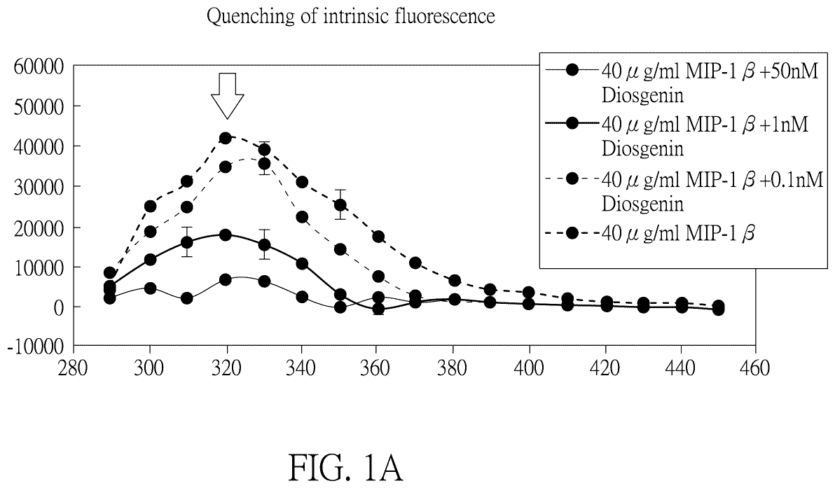

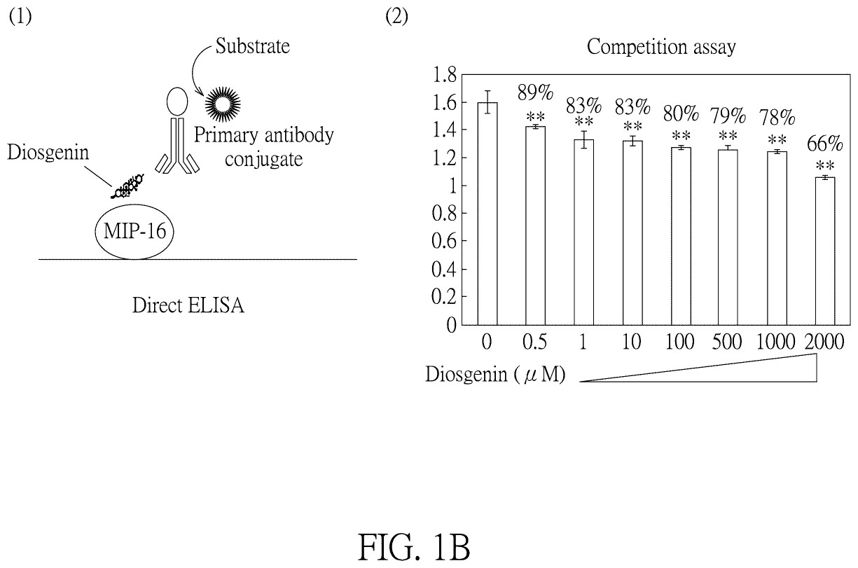

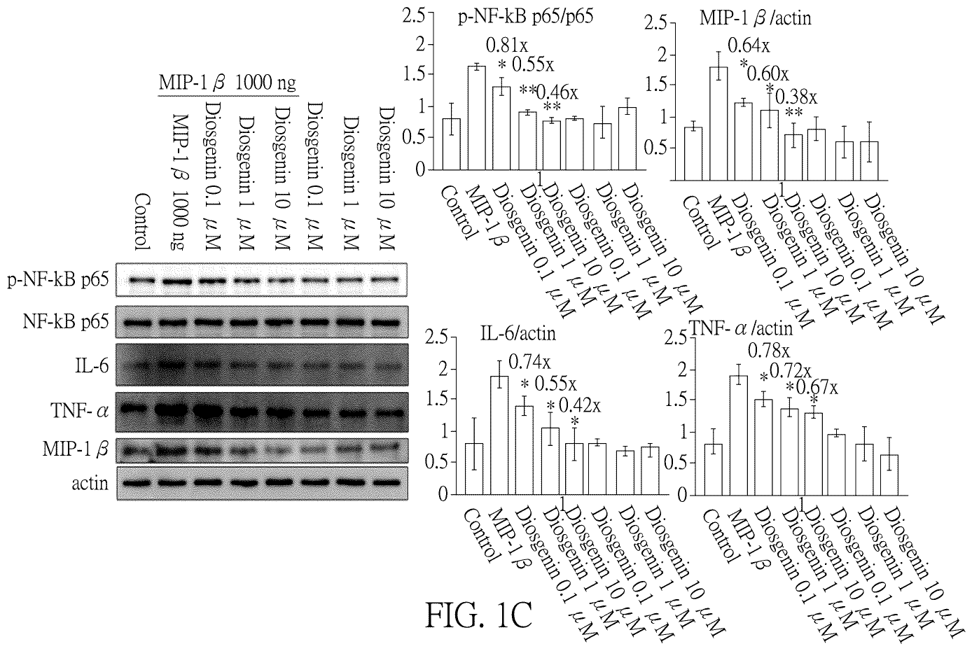

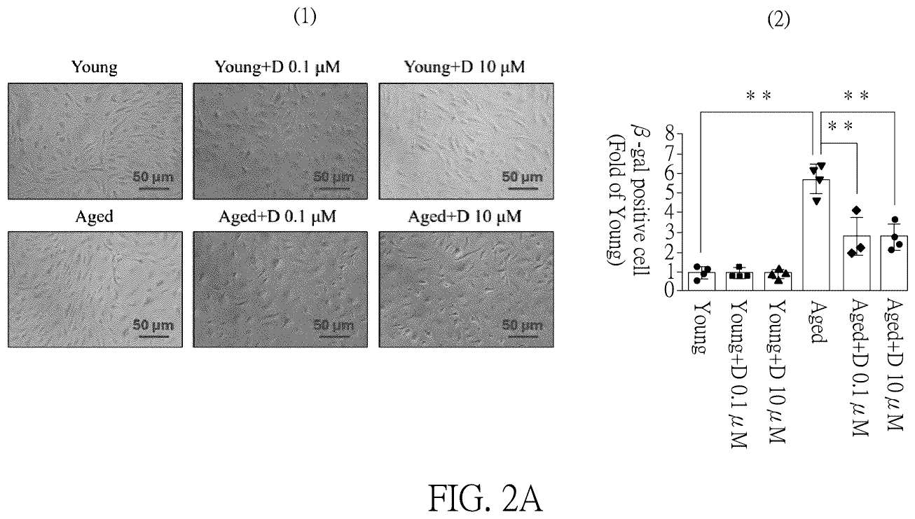

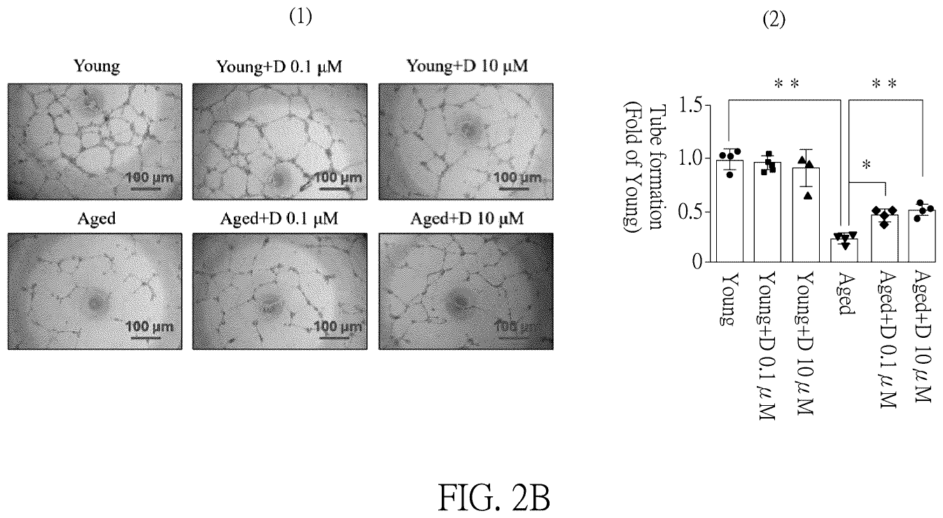

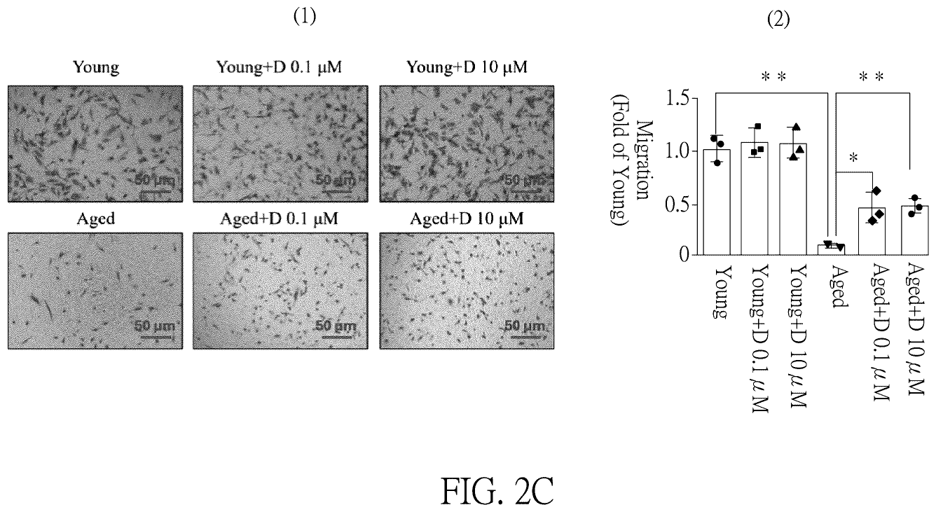

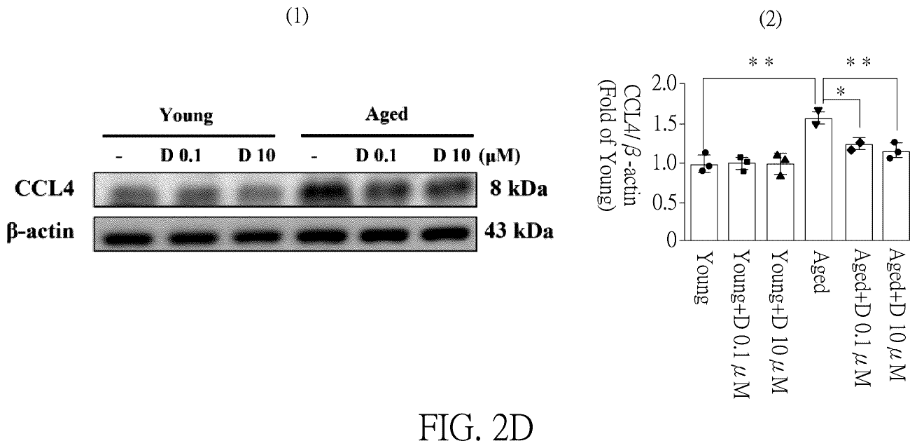

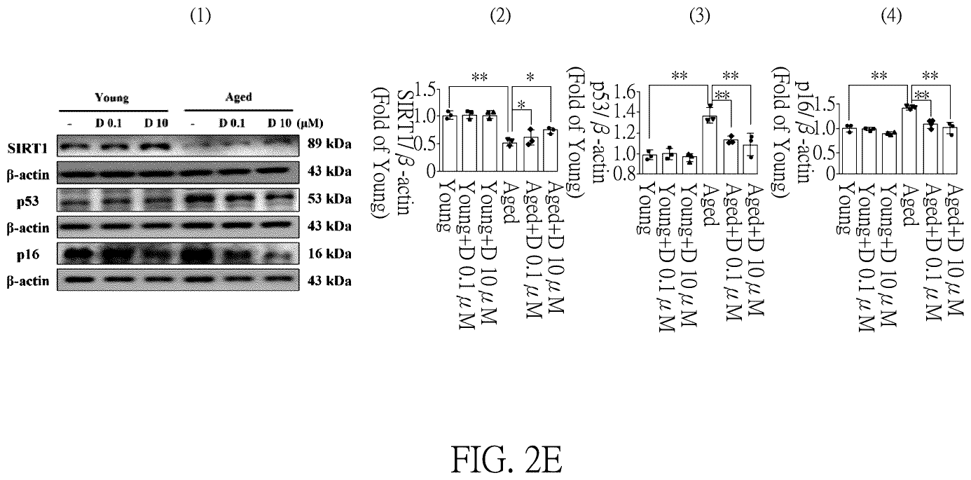

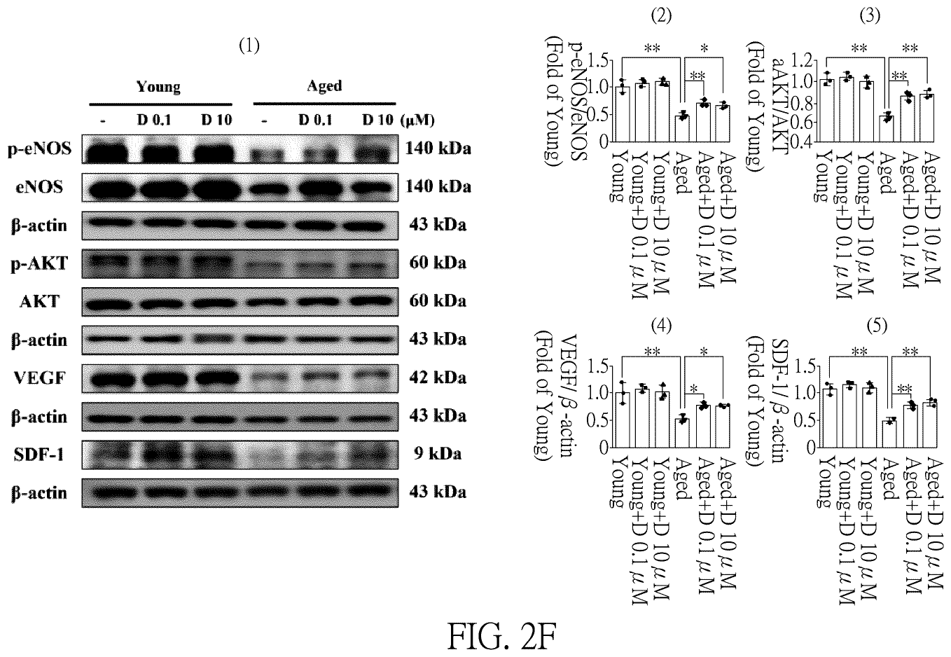

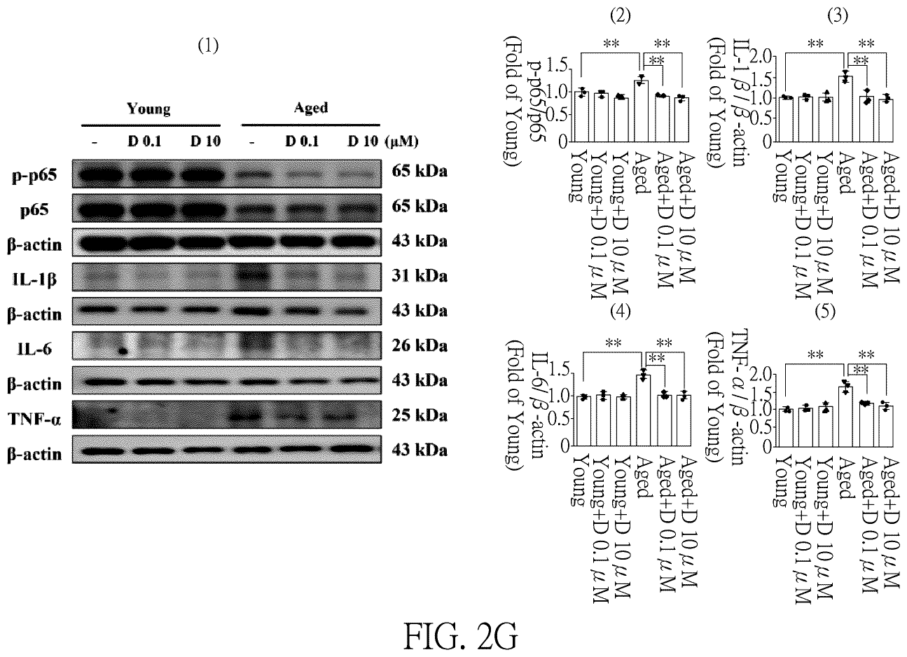

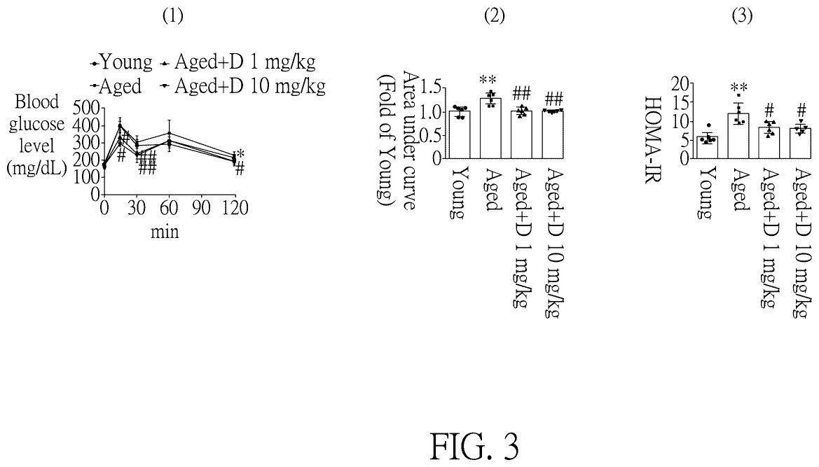

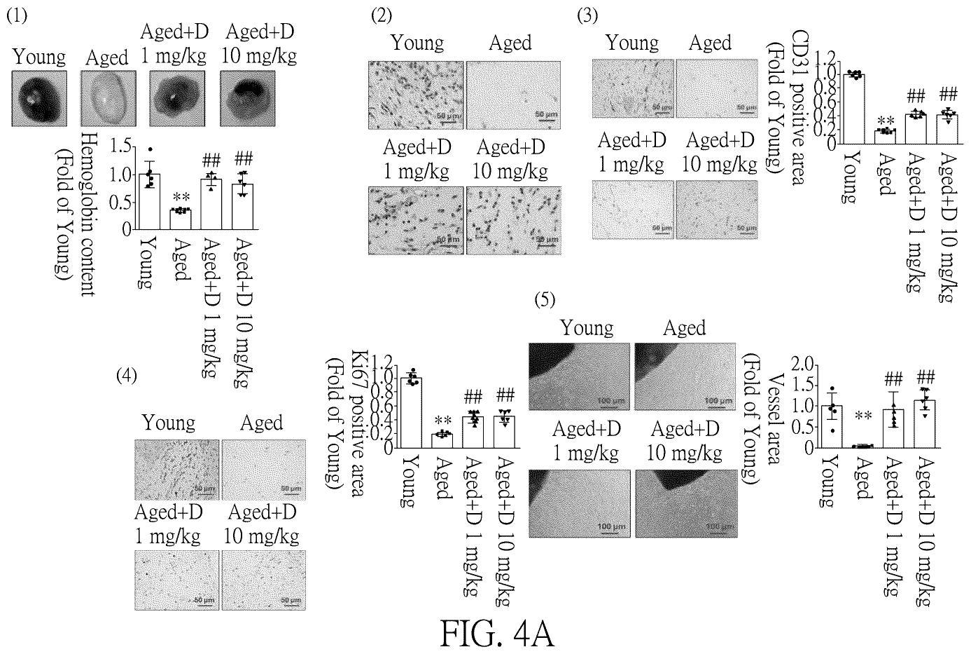

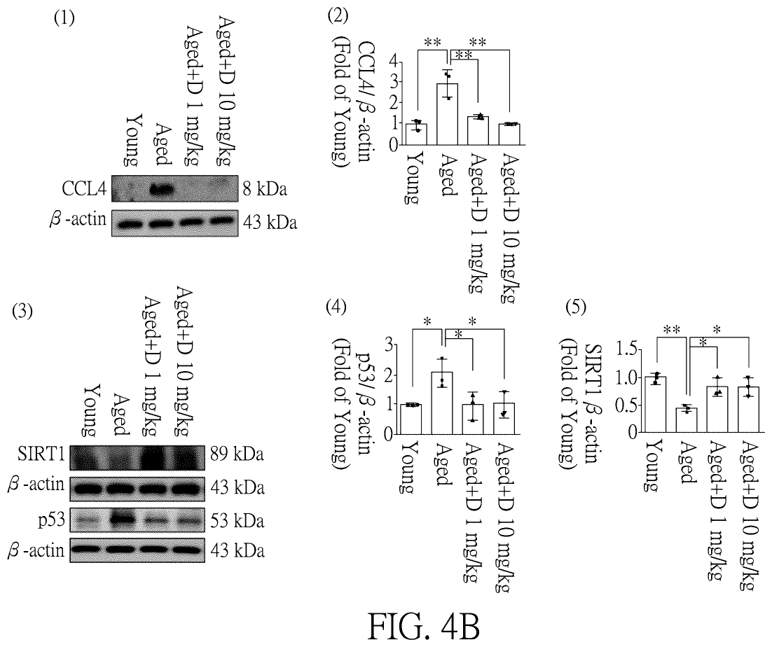

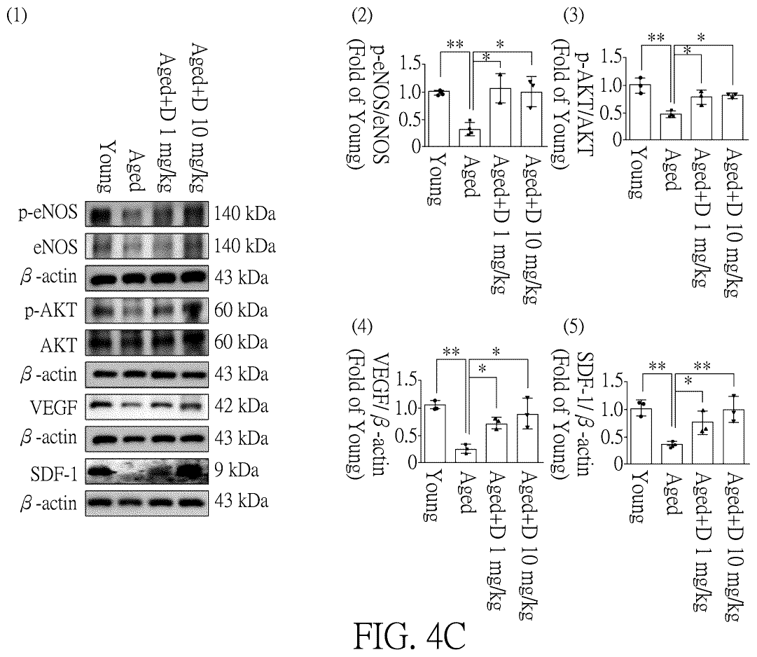

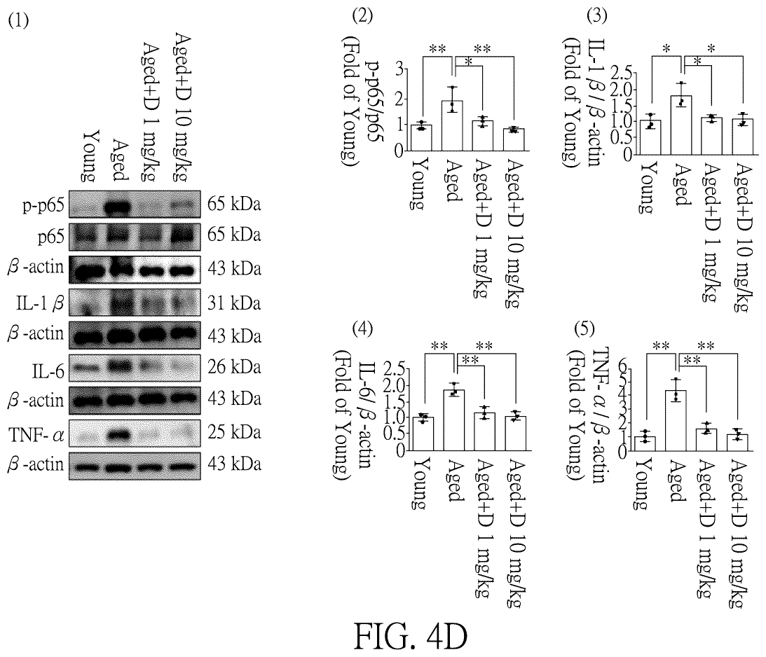

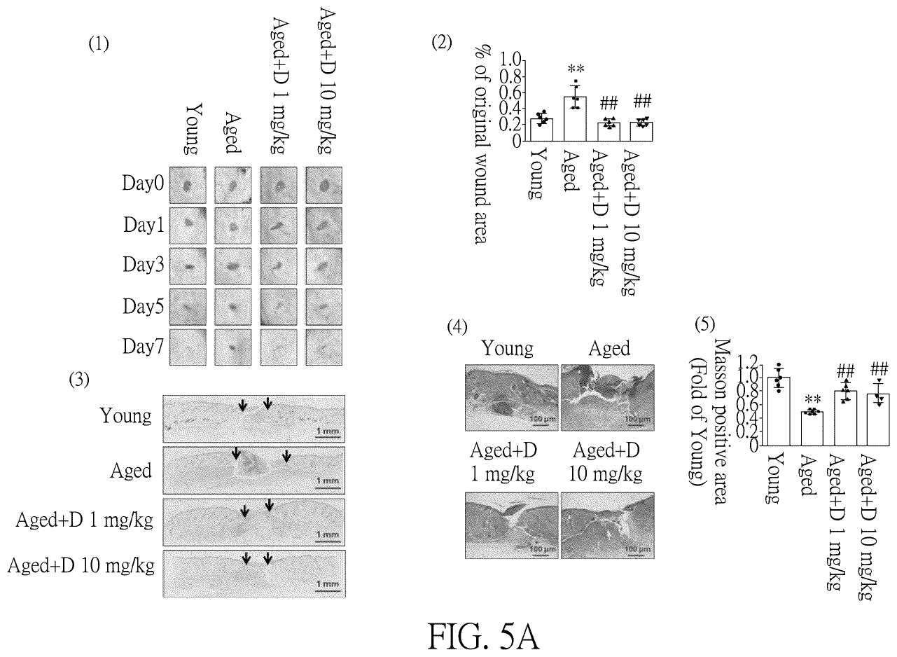

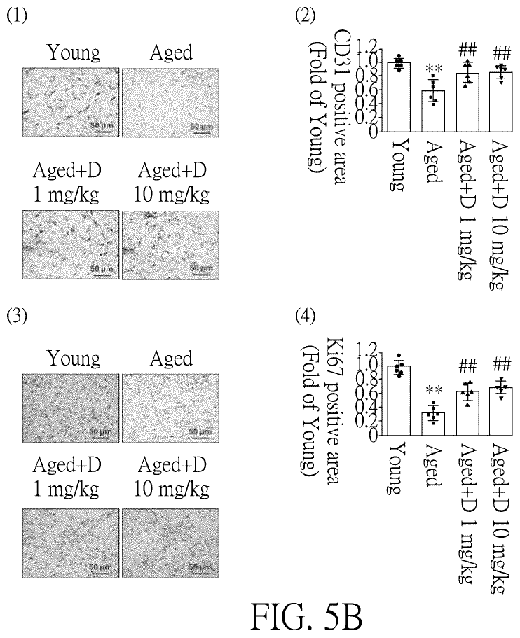

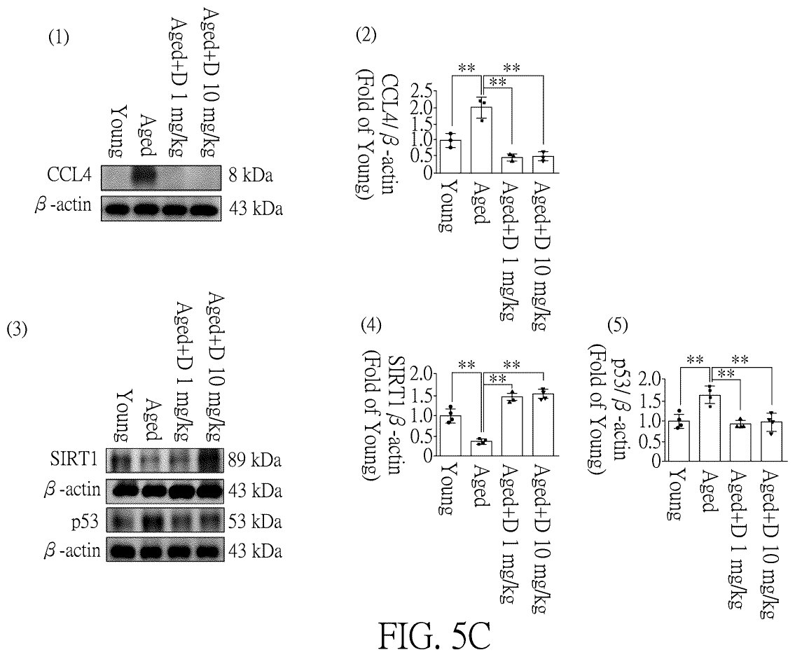

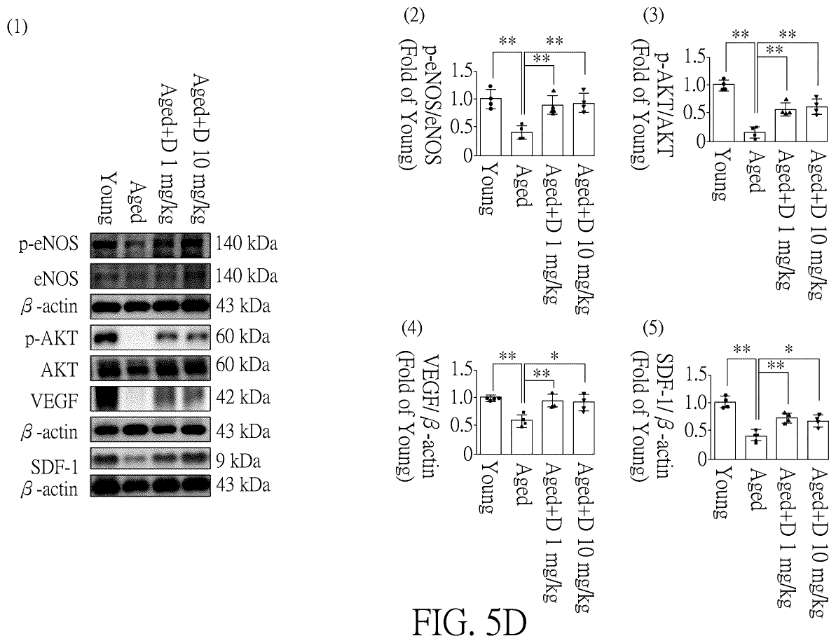

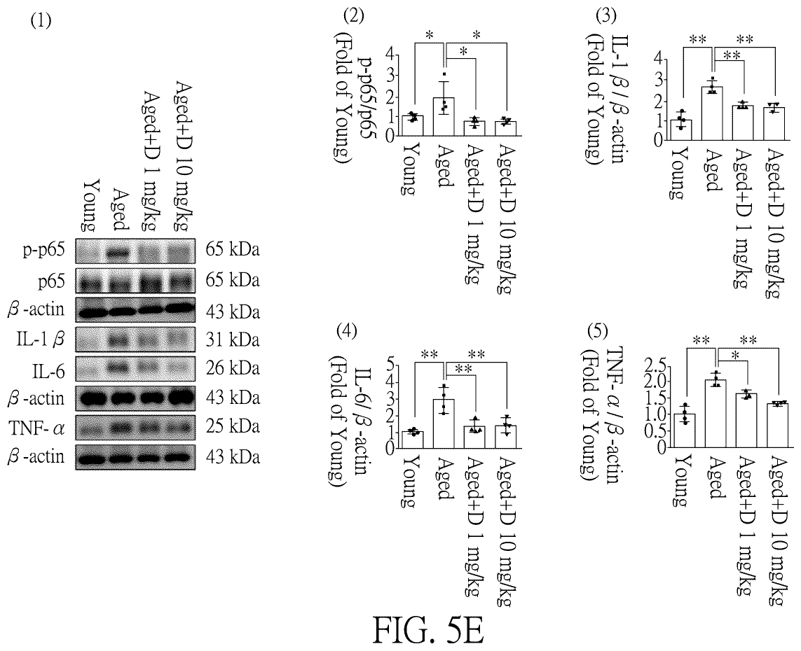

Panel (1) of FIG. 1B and panel (2) of FIG. 1B illustrate diosgenin competition, inhibition of CCL4 antibody by direct enzyme-linked immunosorbent assay (ELISA) according to an embodiment of the present disclosure. * P<0.05, ** P<0.01.

Panel (1) of FIG. 1B and panel (2) of FIG. 1C illustrate western blotting and statistical analyses of p-p65, p65, IL-6, TNF-α, and CCL4 (MIP-1B) according to an embodiment of the present disclosure. * P<0.05, ** P<0.01. Diosgenin administration decreases intercellular TNF-α and IL-6 expression of NIT-1 from exogenous MIP-1ß induction.

Panel (1) of FIG. 2A and panel (2) of FIG. 2A illustrate the beneficial effects of diosgenin on aging-impaired endothelial functions in aged human aortic endothelial cells (HAECs) according to an embodiment of the present disclosure. β-galactosidase positive cells are quantified in HAECs following the diosgenin treatment (n=4). * P<0.05, ** P<0.01. “β-gal” stands for “β-galactosidase” and “D” stands for “diosgenin.” “Young” is short for “young HAECs” and “Aged” is short for “aged HAECs.”

Panel (1) of FIG. 2B and panel (2) of FIG. 2B illustrate the beneficial effects of diosgenin on aging-impaired endothelial functions in aged human aortic endothelial cells (HAECs) according to an embodiment of the present disclosure. Tube branch numbers are quantified after the treatment with diosgenin in HAECs (n=4). * P<0.05, ** P<0.01. “D” stands for “diosgenin.” “Young” is short for “young HAECs” and “Aged” is short for “aged HAECs.”

Panel (1) of FIG. 2C and panel (2) of FIG. 2C illustrate the beneficial effects of diosgenin on aging-impaired endothelial functions in aged human aortic endothelial cells (HAECs) according to an embodiment of the present disclosure. The number of migrated cells is quantified in HAECs following the diosgenin treatment (n=4). * P<0.05, ** P<0.01. “D” stands for “diosgenin.” “Young” is short for “young HAECs” and “Aged” is short for “aged HAECs.”

Panel (1) of FIG. 2D and panel (2) of FIG. 2D illustrate the beneficial effects of diosgenin on aging-impaired endothelial functions in aged human aortic endothelial cells (HAECs) according to an embodiment of the present disclosure. Panel (1) of FIG. 2D shows the western blotting and panel (2) of FIG. 2D shows the statistical analyses of CCL4 (n=3). * P<0.05, ** P<0.01. “D” stands for “diosgenin.” “Young” is short for “young HAECs” and “Aged” is short for “aged HAECs.”

Panels (1) to (4) of FIG. 2E illustrate the beneficial effects of diosgenin on aging-impaired endothelial functions in aged human aortic endothelial cells (HAECs) according to an embodiment of the present disclosure. Panel (1) of FIG. 2E shows the western blotting analyses of SIRT1, p53, and p16 (n=3). Panels (2) to (4) of FIG. 2E shows the statistical analyses of SIRT1, p53, and p16, respectively (n=3). * P<0.05, ** P<0.01. “D” stands for “diosgenin.” “Young” is short for “young HAECs” and “Aged” is short for “aged HAECs.”

Panels (1) to (5) of FIG. 2F illustrate the beneficial effects of diosgenin on aging-impaired endothelial functions in aged human aortic endothelial cells (HAECs) according to an embodiment of the present disclosure. Panel (1) of FIG. 2F shows the western blotting analyses of p-eNOS, p-AKT, VEGF, and SDF-1 (n=3). Panels (2) to (5) of FIG. 2F shows the statistical analyses of p-eNOS, p-AKT, VEGF, and SDF-1, respectively (n=3). * P<0.05, ** P<0.01. “D” stands for “diosgenin.” “Young” is short for “young HAECs” and “Aged” is short for “aged HAECs.”

Panels (1) to (5) of FIG. 2G illustrate the beneficial effects of diosgenin on aging-impaired endothelial functions in aged human aortic endothelial cells (HAECs) according to an embodiment of the present disclosure. Panel (1) of FIG. 2G shows the western blotting analyses of p-p65, IL-1β, IL-6, and TNF-α (n=3). Panels (2) to (5) of FIG. 2G shows the statistical analyses of p-p65, IL-1β, IL-6, and TNF-α, respectively (n=3). * P<0.05, ** P<0.01. “D” stands for “diosgenin.” “Young” is short for “young HAECs” and “Aged” is short for “aged HAECs.”

Panels (1) to (3) of FIG. 3 illustrate the effects of diosgenin on glucose clearance and insulin sensitivity in aged mice according to an embodiment of the present disclosure. Panel (1) of FIG. 3 shows that the oral glucose tolerance test (OGTT) is conducted after diosgenin treatments for 4 weeks (n=6). Panel (2) of FIG. 3 shows that area under curves of the OGTT are calculated from the original graph (n=6). Panel (3) of FIG. 3 shows that insulin levels during the OGTT (n=6). * P<0.05, ** P<0.01 compared with the young group. #P<0.05, ##P<0.01 compared with the aged group. “D” stands for “diosgenin,” and “HOMA-IR” stands for “Homeostatic Model Assessment for Insulin Resistance.” “Young” is short for “young mice” and “Aged” is short for “aged mice.”

Panels (1) to (5) of FIG. 4A illustrate the effects of diosgenin on anti-vascular aging in aged mice according to an embodiment of the present disclosure. Panel (1) of FIG. 4A shows the representative Matrigel plug and analysis of hemoglobin contents (n=6). Panel (2) of FIG. 4A shows the representative images with H&E staining. Panel (3) of FIG. 4A shows the representative images and quantification of CD31 immunostaining in plug sections (n=6). Panel (4) of FIG. 4A shows the representative images and quantification of Ki67 immunostaining in plug sections (n=6). Panel (5) of FIG. 4A shows the representative images and quantification of vessel sprouting aortic rings from mice treatment with diosgenin (n=6). * P<0.05, ** P<0.01 compared with young group. #P<0.05, ##P<0.01 compared with aged group. “D” stands for “diosgenin.” “Young” is short for “young mice” and “Aged” is short for “aged mice.”

Panels (1) to (5) of FIG. 4B illustrate the effects of diosgenin on anti-vascular aging in aged mice according to an embodiment of the present disclosure. Panel (1) of FIG. 4B shows the western blotting analyses of CCL4 (n=3). Panel (2) of FIG. 4B shows the statistical analyses of CCL4 (n=3). Panel (3) of FIG. 4B shows the western blotting analyses of SIRT1 and p53 (n=3). Panels (4) and (5) of FIG. 4B shows the statistical analyses of p53 and SIRT1, respectively (n=3). *P<0.05, ** P<0.01 compared with young group. #P<0.05, ##P<0.01 compared with aged group. “D” stands for “diosgenin.” “Young” is short for “young mice” and “Aged” is short for “aged mice.”

Panels (1) to (5) of FIG. 4C illustrate the effects of diosgenin on anti-vascular aging in aged mice according to an embodiment of the present disclosure. Panel (1) of FIG. 4C shows the western blotting analyses of p-eNOS, p-AKT, VEGF, and SDF-1 (n=3). Panels (2) to (4) of FIG. 4C shows the statistical analyses of p-eNOS, p-AKT, VEGF, and SDF-1, respectively (n=3). *P<0.05, **P<0.01 compared with young group. #P<0.05, ##P<0.01 compared with aged group. “D” stands for “diosgenin.” “Young” is short for “young mice” and “Aged” is short for “aged mice.”

Panels (1) to (5) of FIG. 4D illustrate the effects of diosgenin on anti-vascular aging in aged mice according to an embodiment of the present disclosure. Panel (1) of FIG. 4D shows the western blotting analyses of p-p65, IL-1β, IL-6, and TNF-α (n=3). Panels (2) to (5) of FIG. 4D shows the statistical analyses of p-p65, IL-1β, IL-6, and TNF-α, respectively (n=3). “D” stands for “diosgenin.” “Young” is short for “young mice” and “Aged” is short for “aged mice.”

Panels (1) to (5) of FIG. 5A illustrate the effects of diosgenin on anti-dermal aging in aged mice according to an embodiment of the present disclosure. Panel (1) of FIG. 5A shows the representative wound images. Panel (2) of FIG. 5A shows that the closure rates of 3-millimeter punch biopsies are measured (n=6). Panel (3) of FIG. 5A shows the representative images with H&E staining. Panel (4) of FIG. 5A shows the representative images of collagen positive area in wound sections (n=6). Panel (5) of FIG. 5A shows quantification of collagen positive area in wound sections (n=6). * P<0.05, ** P<0.01 compared with the young group. #P<0.05, ##P<0.01 compared with the aged group. “D” stands for “diosgenin.” “Young” is short for “young mice” and “Aged” is short for “aged mice.”

Panels (1) to (4) of FIG. 5B illustrate the effects of diosgenin on anti-dermal aging in aged mice according to an embodiment of the present disclosure. Panel (1) of FIG. 5B shows the representative images of CD31 immunostaining in wound sections (n=6). Panel (2) of FIG. 5B shows the quantification of CD31 immunostaining in wound sections (n=6). Panel (3) of FIG. 5B shows the representative images Ki67 immunostaining in wound sections (n=6). Panel (4) of FIG. 5B shows the quantification of Ki67 immunostaining in wound sections (n=6). * P<0.05, ** P<0.01 compared with the young group. #P<0.05, ##P<0.01 compared with the aged group. “D” stands for “diosgenin.” “Young” is short for “young mice” and “Aged” is short for “aged mice.”

Panels (1) to (5) of FIG. 5C illustrate the effects of diosgenin on anti-dermal aging in aged mice according to an embodiment of the present disclosure. Panel (1) of FIG. 5C shows the western blotting analyses of CCL4 (n=3). Panel (2) of FIG. 5C shows the statistical analyses of CCL4 (n=3). Panel (3) of FIG. 5C shows the western blotting analyses of SIRT1 and p53 (n=3). Panels (4) and (5) of FIG. 5C show the statistical analyses of SIRT1 and p53, respectively (n=3). *P<0.05, ** P<0.01 compared with the young group. #P<0.05, ##P<0.01 compared with the aged group. “D” stands for “diosgenin.” “Young” is short for “young mice” and “Aged” is short for “aged mice.”

Panels (1) to (5) of FIG. 5D illustrate the effects of diosgenin on anti-dermal aging in aged mice according to an embodiment of the present disclosure. Panel (1) of FIG. 5D shows the western blotting statistical analyses of p-eNOS, p-AKT, VEGF, and SDF-1 (n=3). Panels (2) to (5) of FIG. 5D shows the statistical analyses of p-eNOS, p-AKT, VEGF, and SDF-1, respectively (n=3). * P<0.05, ** P<0.01 compared with the young group. #P<0.05, ##P<0.01 compared with the aged group. “D” stands for “diosgenin.” “Young” is short for “young mice” and “Aged” is short for “aged mice.”

Panels (1) to (5) of FIG. 5E illustrate the effects of diosgenin on anti-dermal aging in aged mice according to an embodiment of the present disclosure. Panel (1) of FIG. 5E shows the western blotting analyses of p-p65, IL-1β, IL-6, and TNF-α (n=3). Panels (2) to (5) of FIG. 5E show the statistical analyses of p-p65, IL-1β, IL-6, and TNF-α, respectively (n=3). “D” stands for “diosgenin.” “Young” is short for “young mice” and “Aged” is short for “aged mice.”

### DETAILED DESCRIPTION

Those skilled in the art will readily observe that numerous modifications and alterations of the present disclosure may be made while retaining the teachings of the disclosure described herein. Accordingly, the embodiments described are intended to cover the modifications and alterations within the scope of the present disclosure, rather than to limit the present disclosure. The scope of the claims therefore should be accorded the broadest interpretation so as to encompass all such modifications and alterations.

In this disclosure, all terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the descriptions of the present disclosure. Thus, the terms used herein are defined based on the meaning of the terms together with the descriptions throughout the specification.

As used herein, the singular forms “a,” “an,” and “the” are intended to include the plural forms, unless the context clearly indicates otherwise. The terms “includes,” “including,” “comprises,” and “comprising” are used in either the detailed descriptions and/or the claims, and such terms are intended to be inclusive in a manner of not excluding others, such as other components, materials, steps, etc. The term “or” is used interchangeably with the term “and/or” unless the context clearly indicates otherwise.

As used herein, the term “comprising,” “comprises” “include,” “including,” “have,” “having,” “contain,” “containing,” and any other variations thereof are intended to cover a non-exclusive inclusion. For example, when describing an object “comprises” a limitation, unless otherwise specified, it may additionally include other ingredients, elements, components, structures, regions, parts, steps, or connections, etc., and should not exclude other limitations.

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of this disclosure, unless the context clearly dictates otherwise.

As used herein, the term “about” generally referring to the numerical value meant to encompass variations of ±20%, ±10%, ±5%, ±1%, ±0.5%, or ±0.1% from a given value or range. Such variations in the numerical value may occur by, e.g., the experimental error, the typical error in measuring or handling procedure for making compounds, compositions, concentrates, or formulations, the differences in the source, manufacture, or purity of starting materials or ingredients used in the present disclosure, or like considerations. Alternatively, the term “about” means within an acceptable standard error of the mean when considered by a person having ordinary skill in the art. Unless otherwise expressly specified, all of the numerical ranges, amounts, values and percentages such as those for quantities of materials, durations of time periods, temperatures, operating conditions, ratios of amounts, and the likes disclosed herein should be understood as modified in all instances by the term “about.”

The numeral ranges used herein are inclusive and combinable, any numeral value that falls within the numeral scope herein could be taken as a maximum or minimum value to derive the sub-ranges therefrom. For example, the numeral range “0.1 μM to 10 μM” includes any sub-ranges between the minimum value of 0.1 μM to the maximum value of 10 μM, such as the sub-ranges from 0.1 μM to 4 μM, from 3 μM to 8 μM, from 1 μM to 9 μM and so on. In addition, a plurality of numeral values used herein can be optionally selected as maximum and minimum values to derive numerical ranges. For instance, the numerical ranges of 0.5 μM to 5 μM, 0.5 μM to 10 μM, and 5 μM to 10 μM can be derived from the numeral values of 0.5 μM, 5 μM, and 10 μM.

As used herein, the phrase “at least one,” in reference to a list of one or more elements, should be understood to mean at least one element selected from any one or more of the elements in the list of elements, but not necessarily including at least one of each and every element listed within the list of elements and not excluding any combinations of elements in the list of elements. This definition also allows that elements may optionally be present other than the elements identified within the list of elements to which the phrase “at least one” refers, whether related or unrelated to those elements identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

As used herein, the term “therapeutically effective amount” refers to the amount of an active agent or a pharmaceutical composition that is sufficient to bring about an effect on treating, preventing, or ameliorating a disorder, disease, or condition of a subject in need thereof. The effective amount may vary by a person ordinarily skilled in the art, depending on excipient usage, routes of administration, the possibility of co-usage with other therapeutic treatment, or the condition to be treated, but the present disclosure is not limited thereto.

As used herein, the term “administer,” “administering” or “administration” refer to the placement of an active ingredient into a subject by a method or route which results in at least partial localization of the active ingredient at a desired site to produce the desired effect. For example, the active ingredient of the present disclosure may be administered to a subject by oral administration, injection, subcutaneous administration, intramuscular administration, topical administration, or nasal administration, but the present disclosure is not limited thereto.

As used herein, the term “treat,” “treating,” or “treatment” refers to obtaining a desired pharmacologic and/or physiologic effect. The effect may be prophylactic in terms of completely or partially preventing a disease or symptoms or conditions thereof or may be therapeutic in terms of completely or partially curing, alleviating, relieving, remedying, or ameliorating a disease or an adverse effect attributable to the disease or symptoms or conditions thereof.

As used herein, the term “prevent,” “preventing,” or “prevention” does not require that the disease state be completely thwarted. Rather, as used herein, the term “prevent,” “preventing,” or “prevention” refers to the ability of the skilled artisan to identify a population that is susceptible to disorders, such that administration of the extracellular vesicles of the present disclosure may occur prior to onset of a disease. The term does not imply that the disease state is completely avoided.

The term “subject” refers to a mammal. The mammal includes, but not limited to, humans, non-human primates, canines, felines, murines, bovines, equines, porcines, sheeps, deers, wolfs, foxes, and rabbits.

The term “diosgenin” as used herein refers to a naturally occurring steroidal sapogenin and includes derivatives, analogs, isomers, solvates, hydrates, esters, prodrugs, polymorphs, and pharmaceutically acceptable salts thereof that retain biological activity associated with inhibition of CCL4 or modulation of inflammatory, angiogenic, metabolic, or senescence-associated pathways. In some embodiments of the present disclosure, diosgenin encompasses synthetic or semi-synthetic derivatives capable of binding CCL4, reducing CCL4 expression, or attenuating CCL4-mediated downstream signaling.

In at least one embodiment of the present disclosure, the effective amount of diosgenin may be about 0.1 mg/kg to about 1000 mg/kg, including but not limited to about 1 mg/kg to about 500 mg/kg. In some embodiments of the present disclosure s, the effective amount may be about 0.1 mg/kg, 0.5 mg/kg, 1 mg/kg, 2 mg/kg, 5 mg/kg, 10 mg/kg, 15 mg/kg, 20 mg/kg, 25 mg/kg, 30 mg/kg, 40 mg/kg, 50 mg/kg, 75 mg/kg, 100 mg/kg, 200 mg/kg, 300 mg/kg, 400 mg/kg, 500 mg/kg, 600 mg/kg, 700 mg/kg, 800 mg/kg, 900 mg/kg, 1000 mg/kg. The appropriate dosage may depend on factors including the age of the subject, metabolic status, severity of wound impairment, degree of inflammatory activation, and route of administration.

In at least one embodiment of the present disclosure, the administration duration of diosgenin or the pharmaceutical composition may range from about 1 day to about 30 days. In some embodiments of the present disclosure, the administration duration may be about 3 days, 5 days, 7 days, 10 days, 14 days, or 21 days, or may continue until substantial wound closure or vascular recovery is achieved. In some embodiments of the present disclosure, repeated or chronic administration may be employed for sustained modulation of inflammatory or metabolic dysfunction.

In at least one embodiment of the present disclosure, the frequency of administration of diosgenin or the pharmaceutical composition may be once daily, twice daily, three times daily, once every two days, once every three days, once weekly, or any clinically appropriate interval. In certain embodiments, dosing frequency is selected to maintain suppression of CCL4 signaling and downstream inflammatory activation.

In at least one embodiment of the present disclosure, diosgenin may be administered prior to wound occurrence, following wound occurrence, or both before and after wound occurrence. In some embodiments of the present disclosure, diosgenin is administered prophylactically in subjects at risk of impaired wound healing due to aging, metabolic syndrome, diabetes, vascular dysfunction, or chronic inflammation.

In some embodiments of the present disclosure, diosgenin may be combined with additional therapeutic agents that promote angiogenesis, improve metabolic control, reduce oxidative stress, or enhance tissue regeneration.

In some embodiments of the present disclosure, the diosgenin may act as a direct inhibitor of chemokine (C-C motif) ligand 4 (CCL4).

In some embodiments of the present disclosure, the aging-related condition may be vascular dysfunction.

In some embodiments of the present disclosure, administration of the diosgenin may increase the expression of at least one angiogenic protein selected from the group consisting of p-eNOS, p-AKT, VEGF, and SDF-1 in an aortic tissue of the subject.

In some embodiments of the present disclosure, the aging-related condition may be dermal dysfunction.

In some embodiments of the present disclosure, administration of the diosgenin may accelerate wound closure and increase collagen deposition, CD31-positive area, and Ki67-positive cells in a wound tissue of the subject.

In some embodiments of the present disclosure, the aging-related condition may be metabolic dysfunction.

In some embodiments of the present disclosure, administration of the diosgenin may improve glucose clearance.

In some embodiments of the present disclosure, administration of the diosgenin may reduce a homeostasis model assessment of insulin resistance (HOMA-IR) level in the subject.

In some embodiments of the present disclosure, administration of the diosgenin may reduce activation of nuclear factor kappa-B (NF-κB) and the expression of at least one inflammatory protein selected from the group consisting of p-p65, IL-1β, IL-6, and TNF-α in the subject.

In some embodiments of the present disclosure, administration of the diosgenin may increase SIRT1 expression and decreases p53 and p16 expression in the subject.

In at least one embodiment of the present disclosure, the therapeutically effective amount of the diosgenin may be from about 0.1 mg/kg/day to about 1000 mg/kg/day.

In at least one embodiment of the present disclosure, the diosgenin may be administered to a subject by oral administration, injection, subcutaneous administration, intramuscular administration, topical administration, or nasal administration, but the present disclosure is not limited thereto.

### Example

Exemplary embodiments according to the present disclosure are further described in the following examples, which should not be construed to limit the scope of the present disclosure. The materials and methods used in the following examples are described in detail below. The materials used in the present disclosure but unannotated herein are commercially available.

### Materials and Methods

### Cell Culture

Primary Human aortic endothelial cells (HAECs; ScienCell, Catalog #6100, Carlsbad, CA, USA) are cultured in fibronectin-coated plates with endothelium cell medium containing 5% fetal bovine serum and 1% of endothelial cell growth supplement in an atmosphere of 95% air and 5% CO2 at 37° C. The young HAECs are at passage 4, and the aged HAECs are at passage 9. Different dosages of diosgenin (0.1, 10 μM; Sigma-Aldrich, D1634, Darmstadt, Germany) are added to the cells as the cells grown to a certain circumstance. The cells are then cultured at 37° C. and 5% CO₂ for 24 hours after diosgenin treatment.

### β-Galactosidase Staining

The senescence phenotype is detected using the β-galactosidase staining kit (Merck, Darmstadt, Germany). The number of positive senescence-associated β-galactosidase cells is observed by light microscopy in 10 randomly chosen low-power fields.

### Migration and Tube Formation Assay

The migration is evaluated by a chamber assay. HAECs (1×104 cells) are resuspended in the culture medium with 5% fetal bovine serum. The cells are added to the upper chamber of a 24-well transwell plate with a polycarbonate membrane. Culture medium supplemented with fetal bovine serum is added to the lower chamber, and the chambers are incubated for 18 hours. After incubation, the membrane is fixed with 4% paraformaldehyde and stained using hematoxylin solution. The numbers of migrated cells are counted in random high-power (×100) microscopic fields.

The in vitro tube formation assay is performed with an angiogenesis assay kit (Invitrogen, Carlsbad, CA, USA). ECMatrix gel solution is mixed with ECMatrix diluent buffer and placed in a 96-well plate. Then, HAECs (1×104 cells) are placed on a matrix solution with 5% fetal bovine serum culture medium and incubated for 16 hours. Tube formation is inspected under an inverted light microscope (×40). The average of the total area of complete tubes formed by cells is compared using Image-Pro Plus (Media Cybernetics, Inc. Rockville, MD, USA).

### Western Blot Analysis

Total cell or tissue lysates are extracted using a lysis buffer, and proteins are separated in 8 to 12% (v/v) SDS-PAGE gels. After electrophoresis (Bio-Rad Laboratories, Hercules, CA, USA), the proteins are transferred onto nitrocellulose membranes (Millipore, Darmstadt, Germany). The membranes are incubated with antibodies against phospho-AKT (BD Biosciences, 550747; NJ, USA); AKT (BD Biosciences, 610868; NJ, USA); Macrophage inflammatory protein-1 (MIP-1B, CCL4; Santa Cruz Biotechnology, sc-130330; Dallas, TX, USA); phospho-endothelial nitric oxide synthase (eNOS; Cell Signaling Technology, 9571S, Boston, MA, USA), eNOS (Cell Signaling Technology, 32027S, Boston, MA, USA); Vascular endothelial growth factor (VEGF; Santa Cruz Biotechnology, sc-152; Dallas, TX, USA); Stromal cell-derived factor-1 (Cell Signaling, 3530S; Boston, MA, USA); Interleukin-1B (IL-1β; Santa Cruz Biotechnology, sc-7884; Dallas, TX, USA); IL-6 (Cell Signaling, 12153S; Boston, MA, USA); Tumor necrosis factor-α (Cell Signaling, 3707S; Boston, MA, USA); phospho-p65 (Cell Signaling, 3031S; Boston, MA, USA); p65 (BD Biosciences, 610868; NJ, USA); Sirtuin (Cell Signaling, 8469S; Boston, MA, USA); p53 (Cell Signaling, 2524S; Boston, MA, USA); p16 (Cell Signaling, 80772S and 29271S; Boston, MA, USA) and actin (Merck, MAB1501, Darmstadt, Germany) at 4° C. overnight. The above protein expressions are normalized to that of actin expression.

### Animal Preparation

Six-month-old C57BL/6JNarl mice are defined as the young group and eighteen-month-old mice are defined as the elderly group. The animals are raised under specific pathogen-free conditions and all mice are kept in microisolator cages on a 12-hour day/night cycle in the animal center of National Yang Ming Chiao Tung University (Taipei, Taiwan) according to the regulations of the Animal Care Committee of National Yang Ming Chiao Tung University. Diosgenin is given once a day by tube feeding at doses of 1 or 10 mg/kg/day for 21 days. The animal study is approved by the Animal Care Committee of National Yang Ming Chiao Tung University (IACUC No. 1101114).

### Wound Healing Assay

The circular full-thickness excisional wounds of 3 mm of diameter are generated with biopsy punch without injuring the muscle. The wounds are recorded using a digital camera (Nikon, Tokyo, Japan) after they are generated. Transverse slices of the wound areas are fixed in 4% paraformaldehyde, embedded in paraffin, and mounted onto slides for staining. Hematoxylin/Eosin stains are used to evaluate the morphological changes in the wound area.

### Histological Analysis

Wounds and plugs are harvested post-sacrificed and fixed in 10% paraformaldehyde before dehydration. The tissues are sectioned into 3 μm thick slides and subjected into hematoxylin and eosin staining. Sections are de-paraffinized and incubated with a rat-monoclonal antibody against murine CD31 (Abcam, 124432, Waltham, MA, USA) and a rabbit-polyclonal antibody against the murine marker of proliferation Ki67 (Novus, NB500-170, Minneapolis, MN, USA). Antibody distribution is visualized with the avidin-biotin-complex technique and Vector Red chromogenic substrate, followed by counterstaining with hematoxylin. Wound sections are allowed to dry overnight and stained with Masson's trichrome-stained for histological analysis.

### Matrigel Plug Assay

Mice are injected subcutaneously with growth factor reduced basement membrane matrix (Corning® Matrigel, Glendale, AZ, USA) containing 30 ng/mL VEGF (Peprotech, Rocky Hill, CT, USA) and 50 U/mL heparin (Sigma-Aldrich, Darmstadt, Germany). Plugs are collected after 14 days and homogenized in 500 μL of cell lysis buffer and centrifuged at 6000 g at 4° C. for 60 minutes. Hemoglobin is detected at 400 nm wavelength by using a colorimetric assay (Sigma-Aldrich, MAK115, Darmstadt, Germany). In addition, the plugs are harvested for histological and immunohistochemistry analysis.

### Aortic Ring Assay

The aortic rings are cut a 0.5 mm and embedded 1 mg/mL type 1 rat tail collagen matrix (Millipore, Darmstadt, Germany) and incubated for 1 hour at 37° C. Aortic rings are cultured in EBM-2 (Lonza, Basel, Switzerland) containing 2.5% bovine serum (Gibco, Carlsbad, CA, USA), 50 U/mL penicillin and 0.5 mg/mL streptomycin (Sigma-Aldrich, Darmstadt, Germany) and 30 ng/mL VEGF (Peprotech, Rocky Hill, CT, USA) in 24-wells for 7 days. Images are captured using a microscope (×100).

### Blood Glucose Test

Mice are fasted for 6 hours, 1 μL of mice blood is collected from the tail. An Abbott FreeStyle glucometer (Abbot-OPTIUM XCEED) is used following the instructions provided by the original manufacturer.

### Oral Glucose Tolerance Tests (OGTTs) and Areas Under the Curves (AUCs)

Mice are fasted for 6 hours and orally administered glucose at a dose of 2 g/kg body weight. Blood samples are obtained at time 0 (just before glucose load) and at 15, 30, 60, 90 and 120 minutes after glucose administration. The total and incremental AUCs for blood glucose during the OGTT are determined by the trapezoidal method.

### Evaluation of Insulin Concentrations

Serum concentrations of insulin are determined by ELISA (Mercodia, 10-1247-01, Uppsala, Sweden) according to the manufacturer's instructions.

### Homeostasis Model Assessment of Insulin Resistance (HOMA-IR)

Insulin resistance, insulin sensitivity, and steady-state β cell function are determined by the HOMA-IR using fasting blood glucose and insulin levels. HOMA analysis is an accepted surrogate for measuring insulin resistance in rodents. The HOMA-IR index is calculated as (fasting insulin (μU/mL)×fasting blood glucose (mmol/L))/22.5.

### Statistical Analysis

The data are analyzed through the Shapiro-Wilk normality test, which is used to determine whether a given data set comes from a normal distribution. In the in vivo and in vitro parts, data are given as the mean±standard deviation, which is based on the parametric nature of the data. The data are analyzed using one-way analysis of variance (ANOVA) followed by post-hoc-Tukey's multiple comparisons test. The results are considered significant at a p value of <0.05. The analyses are performed using SPSS version 29.0 (Chicago, IL, USA).

### Identification of Diosgenin as a Novel Direct CCL4 Inhibitor

Compounds with potential metabolic effects are chosen. Then, docking algorithms are used to predict the interaction between these compounds and CCL4. The compounds are then listed by evaluating their binding affinity with CCL4. Diosgenin, the potential compound with the highest score, is therefore identified and selected for further analysis (Table 1). Computational schematic representation is also used to show the binding site docking of diosgenin with CCL4. Furthermore, the emission spectra of CCL4 are analysis in the presence of diosgenin. The interaction studies reveal that diosgenin may quench the intrinsic fluorescence of CCL4 in a dose dependent manner (FIG. 1A). Diosgenin may also compete with the CCL4 antibody for its inhibition ability to CCL4 in the direct ELISA assay (panels (1) and (2) of FIG. 1B).

TABLE 1

| No. | Names | Docking | Score |
|---|---|---|---|
| 1 | Diosgenin | 1. CCR5 binding site of MIP-1β | 4366 |
| | | 2. Dimer site of MIP-1β | |
| | | 3. Antibody binding site | |
| 2 | Geniposide | 1. CCR5 binding site of MIP-1β | 3658 |
| | | 2. Dimer site of MIP-1β | |
| 3 | Ginsenoside | 1. CCR5 binding site of MIP-1β | 3502 |
| | Rg1 | 2. Dimer site of MIP-1β | |
| | | 3. Antibody binding site | |
| 4 | Dextromethor | 1. CCR5 binding site of MIP-1β | 3274 |
| | phan HBr | 2. Dimer site of MIP-1β | |

To confirm the CCL4 inhibition effects of diosgenin in vitro, NIT-1 cells are used to evaluate the CCL4 inhibition ability of diosgenin on CCL4-related signaling pathway. CCL4 stimulation enhances the inflammatory proteins, such as the activation of nuclear factor kappa-B (NF-κB), interleukin (IL)-6, and tumor necrosis factor-α (TNF-α). The administration of diosgenin may decrease the CCL4-induced cellular inflammation with decreased protein expression of CCL4 (panels (1) and (2) of FIG. 1C). Taken together, these findings suggest that diosgenin can be identified as a novel direct CCL4 inhibitor.

### Diosgenin Reversed Aging-Impaired Endothelial Functions in Aged Human Aortic Endothelial Cells (HAECs)

In vitro experiments are also conducted with HAECs to elucidate the mechanistic role of diosgenin in aging-related vascular dysfunction. The cell function and cell senescence are significantly increased in aged HAECs compared to young HAECs. The administration of diosgenin improves cell function and reduces the senescence of HAECs (FIG. 2A to FIG. 2C). The expression of CCL4 is higher in aged HAECs and is decreased by administration of diosgenin. Administration of diosgenin reverses the aging markers, such as SIRT1, p53, and p16 (FIG. 2D and FIG. 2E). Angiogenic proteins such as p-eNOS, p-AKT, VEGF, and SDF-1 are also enhanced in the diosgenin-treated aged HAECs (FIG. 2F). Moreover, diosgenin reduced the aging-induced inflammatory proteins, such as p-p65, IL-1β, IL-6, and TNF-α (FIG. 2G). Altogether, these findings indicate that diosgenin can restore cell functions with reversed aging factors, enhanced angiogenic factors, and reduced inflammatory factors in aged vascular endothelial cells through its CCL4 inhibition ability.

### Diosgenin Improves Glucose Clearance and Insulin Sensitivity in Aged Mice

In oral glucose tolerance test (OGTT), aged mice treated with diosgenin exhibit lower blood glucose at 15, 30, and 120 minutes compared with that of the untreated aged mice (panel (1) of FIG. 3). The area under the curve during the OGTT is also reduced in diosgenin-treated mice compared with that of the untreated aged mice (panel (2) of FIG. 3). In addition, the administration of diosgenin can lower the levels of homeostasis model assessment of insulin resistance (HOMA-IR) (panel (3) of FIG. 3). These results suggest that diosgenin can improve glucose clearance and insulin sensitivity in aged mice.

### Diosgenin Facilitates Angiogenesis and Reversed Vascular Aging in Aged Mice

In Matrigel plug angiogenesis assay, higher vessel formation and hemoglobin content are observed in the plugs from aged mice treated with diosgenin (panel (1) of FIG. 4A). Enhanced neovascularization is observed in diosgenin-treated aged mice, as shown by Matrigel plug sections stained with hematoxylin and eosin (H&E) (panel (2) of FIG. 4A). The enhanced CD31 and Ki67 expressions are also detected in the Matrigel plug sections from diosgenin-treated aged mice (panels (3) and (4) of FIG. 4A). In addition, aortic sprouting is impaired in aortic rings from the untreated aged mice compared to those from young mice. Aortic rings from aged mice treated with diosgenin have a greater vessel sprouting number compared to those from untreated aged mice (panel (5) of FIG. 4A). The aortic expression of CCL4 is higher in aged mice and is decreased by the administration of diosgenin (panels (1) and (2) of FIG. 4B). Administration of diosgenin also reverses the aging markers, such as SIRT1 and p53 (panels (1), (2) and (3) of FIG. 4B). Angiogenic proteins, such as p-eNOS, p-AKT, VEGF, and SDF-1 are also enhanced in the aortic tissues from diosgenin-treated aged mice (panels (1) to (5) of FIG. 4C). Moreover, diosgenin reduces the age-induced inflammatory proteins, such as p-p65, IL-1β, IL-6, and TNF-α in the aorta (panels (1) to (5) of FIG. 4D). Therefore, CCL4 inhibition by diosgenin exerts neovascularization and anti-vascular aging effects in aged mice.

### Diosgenin Accelerated Wound Repair and Reversed Dermal Aging in Aged Mice

In the wound healing assay, the aged mice present delayed wound repair post-injury compared to that of the young group. The aged mice treated with diosgenin show an accelerated rate of wound closure compared to the untreated aged mice (panels (1) and (2) of FIG. 5A). Improved wound repair is observed in diosgenin-treated aged mice, as shown in wound sections stained with H&E (panel (3) of FIG. 5A). Collagen deposition is increased in diosgenin-treated aged mice compared with that of the untreated aged mice (panels (4) and (5) of FIG. 5A). Higher CD31 and Ki67 expressions in the wound sections were detected in diosgenin-treated mice, as compared with the untreated aged mice (panels (1) to (4) FIG. 5B). The expression of CCL4 is higher in aged mice and is decreased by the administration of diosgenin in the wound tissues (panels (1) and (2) of FIG. 5C). In addition, treatment with diosgenin reverse the aging markers, such as SIRT1 and p53 in wound tissues (panels (3) to (5) FIG. 5C). Angiogenic proteins, such as p-eNOS, p-AKT, VEGF, and SDF-1 are also increased in the wound tissues of diosgenin-treated aged mice (panels (1) to (5) FIG. 5D). Furthermore, diosgenin decreases the age-induced inflammatory proteins, such as p-p65, IL-1β, IL-6, and TNF-α in the wound tissues (panels (1) to (5) FIG. 5E). The above findings indicate that the inhibitory effect of diosgenin on CCL4 can promote wound repair and anti-skin aging in aged mice.

The foregoing outlines the features of several embodiments, enabling those skilled in the art to fully appreciate the aspects of the present disclosure. Those skilled in the art should recognize that the present disclosure provides a foundation for designing or modifying other processes and structures to achieve substantially the same functions and/or substantially the same results as those of the embodiments introduced herein. Furthermore, such equivalent arrangements do not deviate from the spirit and scope of the present disclosure, and various changes, substitutions, and alterations may be made without so departing.

What is claimed is:

1. A method for treating or preventing an aging-related condition, comprising:
administering to a subject in need thereof a therapeutically effective amount of diosgenin, wherein the aging-related condition is selected from the group consisting of vascular dysfunction, dermal dysfunction, and metabolic dysfunction.

2. The method of claim 1, wherein the diosgenin acts as a direct inhibitor of chemokine (C-C motif) ligand 4 (CCL4).

3. The method of claim 1, wherein the aging-related condition is vascular dysfunction.

4. The method of claim 3, wherein administration of the diosgenin increases the expression of at least one angiogenic protein selected from the group consisting of p-eNOS, p-AKT, VEGF, and SDF-1 in an aortic tissue of the subject.

5. The method of claim 1, wherein the aging-related condition is dermal dysfunction.

6. The method of claim 5, wherein administration of the diosgenin accelerates wound closure and increases collagen deposition, CD31-positive area, and Ki67-positive cells in a wound tissue of the subject.

7. The method of claim 1, wherein the aging-related condition is metabolic dysfunction.

8. The method of claim 7, wherein administration of the diosgenin improves glucose clearance.

9. The method of claim 8, wherein administration of the diosgenin reduces a homeostasis model assessment of insulin resistance (HOMA-IR) level in the subject.

10. The method of claim 1, wherein administration of the diosgenin reduces activation of nuclear factor kappa-B (NF-κB) and the expression of at least one inflammatory protein selected from the group consisting of p-p65, IL-1β, IL-6, and TNF-α in the subject.

11. The method of claim 1, wherein administration of the diosgenin increases SIRT1 expression and decreases p53 and p16 expression in the subject.
